(19) United States
(12) Patent Application Publication
(10) Pub. No.: US 2006/0217445 A1
(43) Pub. Date: Sep. 28, 2006

(54) Natural astaxanthin extract reduces dna oxidation

(76) Inventors: Boon P. Chew, Pullman, WA (US); Jean Soon Park, Pullman, WA (US)

Correspondence Address: KLARQUIST SPARKMAN, LLP, 121 SW SALMON STREET, SUITE 1600, PORTLAND, OR, 97204, US

(21) Appl. No.: 10/565,717

(22) PCT Filed: Jul. 26, 2004
(86) PCT No.: PCT/US04/24314

§ 371 Date: Jan. 23, 2006

Related U.S. Application Data
(60) Provisional application No. 60/490,121, filed on Jul. 25, 2003.

(51) Int. Cl. A61K 31/12; A61K 31/015

(52) U.S. Cl. 514/690; 514/763; 514/560

(57) ABSTRACT

Provided herein are methods for reducing oxidative DNA damage in a subject, by administering to the subject astaxanthin, for instance a natural, astaxanthin-enriched extract from Haematococcus pluvialis. It is shown that doses as low as 2 mg/day, given orally to a human subject for a period of four weeks, is sufficient to reduced measurable endogenous oxidative DNA damage by about 40%.

### CROSS REFERENCE TO RELATED APPLICATION

This application claims the benefit of U.S. Provisional Patent Application No. 60/490,121, filed on Jul. 25, 2003, which is incorporated herein by reference in its entirety.

### FIELD OF THE DISCLOSURE

This disclosure is related to reducing oxidative damage to DNA in cells, particularly in immune cells in mammals. More specifically, this disclosure provides methods of using a natural extract comprising and enriched in astaxanthin to reduce, prevent, or treat oxidative damage to DNA in mammals.

### BACKGROUND

The molecular reduction of oxygen to water during oxidative phosphorylation results inevitably in the production of superoxide radicals (.O₂) that are reactive oxygen species containing an unpaired electron orbital. Superoxides act as either reductants or oxidants and can form other reactive species including the hydroxyl radical (.OH) through interaction with iron (Haber-Weiss reaction) and peroxynitrite by reaction with nitric oxide. Reactive oxygen species attack proteins, DNA, and membrane lipids, thereby disrupting cellular function and integrity.

It has long been believed that oxidative damage to cells, tissues, and genetic material, plays a major role in aging and illness. Sources of oxidative damage are many, and include chemicals present in the environment, aging, disease, intense exercise, and ionizing radiation. Additionally, many products and byproducts of cellular metabolism can cause or contribute to oxidative damage.

The immune system is a key player in defense against disease and cancer. Unfortunately, the immune system is particularly susceptible to oxidative damage. Immune cells are highly active cells, which undergo rapid division, especially when challenged. The cellular membranes of immune cells contain a high percentage of polyunsaturated fatty acids. Immune cells also generate reactive and highly reactive oxidative agents, which are part of an arsenal used to attack and neutralize various challenges encountered as part of their normal immune activity.

Even though mammals produce a number of antioxidant enzymes, these enzymes are often insufficient to adequately eliminate oxidative agents; conditions of heightened oxidative stress only make matters worse. Dietary supplementation with antioxidants can be particularly useful in lessening the damage caused by any oxidative agents.

Among the most potent antioxidants known are the carotenoids. This family of compounds includes both carotenes such as β-carotene, and xanthophylls such as lutein, lycopene and astaxanthin. Carotenoids work to remove oxidative agents primarily by quenching singlet oxygen and scavenging free radicals to prevent and terminate chain reactions. Astaxanthin is particularly potent in quenching singlet oxygen, and has over five hundred times the ability to quench singlet oxygen as α-tocopherol. It has a unique molecular structure that gives it powerful antioxidant function. It is extracted from salmon, crustaceans, microalgae, and Phaffia (a yeast, also known as Pfaffia), and it can be chemically synthesized.

### SUMMARY OF THE DISCLOSURE

It has surprisingly been found that oxidative DNA damage (as measured, for instance, by level of 8-OHdG) can be significantly reduced in a subject, by administering low dosages of a natural astaxanthin enriched extract to the subject. This effect was shown with levels as low as 2 mg/day of astaxanthin (administered orally, in the form of a natural astaxanthin enriched extract from Haematococcus pluvialis), after as little as four weeks of administration. The reduction in 8-OHdG (measured as gm/mL) was as much as 60%, compared to both the same subject before administration, and to a subject not provided the natural astaxanthin enriched extract.

The present disclosure therefore provides a method for reducing or inhibiting oxidative DNA damage in a subject, by providing the subject with a therapeutically effective dose of astaxanthin. Oral administration is contemplated, for instance in the form of a capsule, tablet, or pill comprising astaxanthin, particularly naturally occurring esterified astaxanthin. It may also be combined with other constituents, such as other antioxidants, vitamins, minerals, drugs, etc. Intravenous administration is also contemplated, for instance when oral administration would not be applicable. It is also contemplated that the astaxanthin can be administered to a subject in or accompanied by a food or beverage substance.

Provided herein is a method of reducing, preventing, ameliorating, or reversing oxidative DNA damage in a subject (such as a human subject), which method involves orally administering a therapeutically effective dose of a natural astaxanthin extract to the subject, whereby the natural astaxanthin extract reduces, prevents, ameliorates, or reverses the oxidative DNA damage. In certain examples of the method, the natural astaxanthin extract comprises predominantly mono- and di-ester forms of astaxanthin. For instance, in specific embodiments, the natural astaxanthin extract comprises no more than about 5% free astaxanthin, about 45-50% astaxanthin monoesters, about 10-40% astaxanthin diesters, and other carotenoids in the remaining percentage. By way of example, the other carotenoids may be β-carotene, lutein, canthaxanthin, or a mixture of two or more thereof.

It is contemplated in examples of the provided method, the natural astaxanthin extract is derived from yeast (such as a Phaffia species) or microalgae (such as Haematococcus pluvialis).

In examples of the method, the astaxanthin in the extract is greater than 95% (3S,3′S) astaxanthin, for instance, as much as about 100% (3S,3′S) astaxanthin. The astaxanthin in the extract in some embodiments comprises about 55-62% E-astaxanthin, about 13-18% 9Z-astaxanthin, and about 23-29% 13Z-astxanthin. Optionally, natural astaxanthin extract used in the methods described herein further comprises fatty acids, and the fatty acids are one or more of Lauric, Tridecanoic, Myristic, Pentadecanoic, Palmitic, cis-9-Palmitoleic, Heptadecanoic, cis-10-Heptadecenoic, Stearic, cis-9-Oleic and/or trans-9-Elaidic, cis-9,12-Linoleic and/or trans-9,12-Linolelaidic, Arachidic, alpha-Linolenic, cis-11-Eicosenoic, Linolenic, Heneicosanoic, cis-11,14-Eicosadienoic, Behenic, cis-8,11,14-Eicosatrienoic, cis-13-Erucic, cis-11,14,17-Eicosatrienoic, cis-5,8,11,14-Arachidonic, and cis-5,8,11,14,17-Eicosapentaenoic acids.

Examples of the natural astaxanthin extract are produced by a process comprising supercritical carbon dioxide extraction, particularly supercritical carbon dioxide extraction without the addition of other chemicals that might remain in the extract as contaminants.

In various embodiments of the provided method for reducing, preventing, ameliorating, or reversing oxidative DNA damage in a subject, the natural astaxanthin extract is administered to the subject in combination with (either concurrently or in sequence) at least one additional biologically active compound. By way of example, the biologically active compound is a carotenoid, an antioxidant, a vitamin, or a second natural extract.

In various embodiments of the described methods, the natural astaxanthin extract is dissolved in oil; dispersed in oil; dispersed in an aqueous medium; homogenized in an aqueous medium; encapsulated; processed into dry material (such as stabilized beadlets, a powder, a granule, or a combination of two or more thereof); or a combination of two or more thereof. By way of specific example, the natural astaxanthin extract is formulated as a liquid, a liquid capsule, a solid capsule or a tablet. Optionally, the natural antioxidant extract is administered to the subject in or with a food or beverage product.

In various embodiments, the therapeutically effective dose astaxanthin reduces the oxidative DNA damage by at least 30%, compared to a subject not administered the therapeutically effective dose of astaxanthin.

Examples of the provided methods are beneficial in that they are effective for reducing, preventing, ameliorating, or reversing oxidative DNA damage in immune cells in the subject. By way of example, the immune cells are cells, B-cells, monocytes, neutrophils, natural killer cells, splenocytes, or a mixture of two or more thereof.

Therapeutically effective doses in the described methods will vary, but in general an effective dose is about 0.5-1000 mg astaxanthin per day, and most often about 1-10 mg per day. In specific embodiments, the therapeutically effective dose is about 2 mg per day, about 4 mg per day, or about 8 mg per day.

The foregoing and other features and advantages will become more apparent from the following detailed description of several embodiments, which proceeds with reference to the accompanying figures.

### BRIEF DESCRIPTION OF THE DRAWINGS

FIG. 1 is a graph showing concentrations of plasma astaxanthin in human subjects fed 0, 2 or 8 mg astaxanthin daily for 8 weeks.

FIG. 2 is a graph showing the response to phytohemagglutinin-induced lymphocyte proliferation in human subjects fed 0, 2 or 8 mg astaxanthin daily for 8 weeks.

FIG. 3 is a graph showing the response to concanavalin A-induced lymphocyte proliferation in human subjects fed 0, 2 or 8 mg astaxanthin daily for 8 weeks.

FIG. 4 is a graph showing the response to pokeweed mitogen-induced lymphocyte proliferation in human subjects fed 0, 2 or 8 mg astaxanthin daily for 8 weeks.

FIG. 5 is a graph showing the natural killer cell cytotoxic activity (1:10) in human subjects fed 0, 2 or 8 mg astaxanthin daily for 8 weeks.

FIG. 6 is a graph showing the percent of total T cells in blood from human subjects fed 0, 2 or 8 mg astaxanthin daily for 8 weeks.

FIG. 7 is a graph showing the percent of B cells in blood from human subjects fed 0, 2 or 8 mg astaxanthin daily for 8 weeks.

FIG. 8 is a graph showing the percent of LFA-1+ (adhesion molecule) cells in blood from human subjects fed 0, 2 or 8 mg astaxanthin daily for 8 wk.

FIG. 9 is a graph showing the response to the delayed type hypersensitivity tuberculin test in human subjects fed 0, 2 or 8 mg astaxanthin daily for 8 weeks.

FIG. 10 is a graph showing concentrations of plasma 8-OHdeoxyguanosine in human subjects fed 0, 2 or 8 mg astaxanthin daily for 8 weeks.

FIG. 11 is a graph showing concentrations of plasma 8-isoprostane in human subjects fed 0, 2 or 8 mg astaxanthin daily for 8 weeks.

In all of the figures, letter notations above certain bars indicate matched statistical significance within that experiment. Thus, two bars that are marked with the same letter (e.g., “a”) are statistically different from each other at a confidence level of greater than 0.05 (p<0.05). Bars marked with different letters are not statistically different at that confidence level.

### DETAILED DESCRIPTION

### I. ABBREVIATIONS

8-OHdG 8-OHdeoxyguanosine

CO₂ carbon dioxide

DNA deoxyribonucleic acid

ECD electrochemical detection

FPG formamidopyrmidine glycosylase

GC gas chromatography

HDPE high density polyethylene

HPLC high performance lipid chromatography

MS mass spectrometry

QSAR quantitative structure activity relationships

ROS reactive oxygen species

SCFE supercritical fluid extraction

SFE supercritical fluid extraction

### II. TERMS

Unless otherwise noted, technical terms are used according to conventional usage. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

In order to facilitate review of the various embodiments of the invention, the following explanations of specific terms are provided:

Antioxidant: A substance that, when present in a mixture containing an oxidizable substrate biological molecule, significantly delays, reduces, reverses or prevents oxidation of the substrate biological molecule. Antioxidants can act by scavenging biologically important reactive free radicals or other reactive oxygen species (.O₂⁻, H₂O₂, .OH, HOCl, ferryl, peroxyl, peroxynitrite, and alkoxyl), or by preventing their formation, or by catalytically converting the free radical or other reactive oxygen species to a less reactive species.

Astaxanthin: A carotenoid with a unique molecular structure that gives it powerful antioxidant function. Astaxanthin is well known as the pigment providing the pinkish-red hue to the flesh of salmon and trout, as well the coloring in the carapaces of shrimp, lobsters and crayfish.

The astaxanthin molecule has two asymmetric carbons located at the 3 and 3′ positions of the benzenoid rings on either end of the molecule. Different enantiomers of the molecule result from the exact way that the hydroxyl groups (—OH) are attached to the carbon atoms at these centers of asymmetry. When the hydroxyl group is attached so that it projects above the plane of the molecule, it is said to be in the R configuration; when the hydroxyl group projects below the plane of the molecule, it is said to be in the S configuration. Thus the three possible enantiomers of astaxanthin are designated (3R,3′R), (3S,3′S) and (3R,3′S; meso).

Free astaxanthin and its mono- and diesters from Haematococcus have optically pure (3S,3′S)-chirality. Only the (3S,3′S) isomer of astaxanthin is found in the skin and flesh of some salmonid fish. Salmonids are unable to epimerize the 3-hydroxy groups, so it is believed that their dietary carotenoid is also 3S,3′S-astaxanthin. This is consistent other studies (e.g. Storebakken et al., Aquaculture 44:259-269, 1984), where the same chiral composition of astaxanthin found in the crustaceans as in the fishes Salvelinus alpinus and Salmo trutta. Another study revealed that fish caught from the wild in Scotland, Ireland and Norway contained greater 80% 3S, 3′S astaxanthin in the flesh (Schiedt et al., Helv. Chim. ACTA 64:449-457, 1981). HPLC separation of astaxanthin has been used to identify the eggs of escaped salmon, since wild fish contain about 80% astaxanthin and farmed fish fed chemically synthesized astaxanthin contain 35% or less (Lura & Saegrov, Can. J. Fish. Aquat. Sci. 48:429-433, 1991; Turujman et al., J. AOAC Int. 80:622-632, 1997). The chirality of astaxanthin is believed to influence biological functions of this carotenoid.

Damage: Any damage resulting from a variety of oxidative agents such as oxygen itself, hydroxyl radical, hydrogen peroxide, other free radicals, ozone etc., or from any kind of harmful irradiation, such as alpha, beta or gamma rays, neutron radiation, and UVA and UVB irradiation.

Enantiomers: Enantiomers are forms of a molecule that exist as non-superimposable mirror images of one another. Not being able to superimpose one molecule form on top of the other simply means that the two are not equivalent or identical. For a compound to form an enantiomeric pair, it must have chiral molecules. Chiral molecules must not have an internal plane of symmetry, and they must have a stereocenter. Enantiomers are also called optical isomers because their solutions rotate the plane of polarized light passing through them. If one enantiomer rotates light in the clockwise direction, a solution of the other enantiomer will rotate it in the opposite direction.

Another way to characterize enantiomers is by their configuration. Configuration is the spatial way that non-equivalent groups arrange themselves around a stereocenter carbon. One enantiomer will be configured right handedly (R; rectus) and the other will be configured left handedly (S; sinister). Enantiomers are usually depicted on a planar surface either as a 3-dimensional structural formula or as a Fisher Projection.

Free Radicals: Atoms, ions or molecules that contain an unpaired electron. Free radicals are usually unstable, and have short half-lives. Reactive oxygen species (ROS) is a collective term, designating the oxygen radicals (such as the .O₂⁻superoxide radical), which by sequential univalent reduction produces hydrogen peroxide (H₂O₂) and hydroxyl radical (.OH). The hydroxyl radical sets 20 off chain reactions and can interact with nucleic acids. Other ROS include nitric oxide (NO.) and peroxy nitrite (NOO.), and other peroxyl (RO₂.) and alkoxyl (RO.) radicals. Increased production of these poisonous metabolites in certain pathological conditions is believed to cause cellular damage through the action of the highly reactive molecules on proteins, lipids and DNA. In particular, ROS are believed to accumulate when tissues are subjected to ischemia, particularly when followed by reperfusion.

Molecular oxygen is essential for aerobic organisms, where it participates in many biochemical reactions, including its role as the terminal electron acceptor in oxidative phosphorylation. Excessive concentrations of various forms of reactive oxygen species and other free radicals can have serious adverse biological consequences, including the peroxidation of membrane lipids, hydroxylation of nucleic acid bases, and the oxidation of sulfhydryl groups and other protein moieties. Biological antioxidants include tocopherols and tocotrieneols, carotenoids, quinones, bilirubin, ascorbic acid, uric acid, and metal binding proteins. These endogenous antioxidant systems are often overwhelmed by pathological processes that allow permanent oxidative damage to occur to tissue.

Injectable Composition: A pharmaceutically acceptable fluid composition comprising at least an active ingredient. The active ingredient is usually dissolved, disseminated, or suspended in a physiologically acceptable carrier, and the composition can additionally comprise minor amounts of one or more non-toxic auxiliary substances, such as emulsifying agents, preservatives, and pH buffering agents and the like. Such injectable compositions that are useful for use with the natural astaxanthin extracts used in methods of this invention are conventional; appropriate formulations are well known in the art.

Natural Astaxanthin Extract: An oily, viscous dark red lipophilic extract of an organism that comprises, and preferably produces, astaxanthin, particularly an astaxanthin-rich organism (e.g., Phaffia spp., Haematococcus spp.), which extract contains free astaxanthin, astaxanthin fatty acid mono-esters and astaxanthin fatty acid di-esters along with triglycerides and other lipophilic compounds. Carotenoid pigments found from different sources of Haematococcus pluvialis have been found to have the following typical ranges: Astaxanthin (total) 81-99% (which comprises free astaxanthin 1-5%; astaxanthin monoesters 46-79%; astaxanthin diesters 10-39%); β-carotene 0-5%; lutein 1-11%; canthaxanthin 0-5.5%; and other carotenoids 1-9% (Renstrom et al., Phytochemistry 20:2561-2564, 1981; Aquasearch, FDA 75-day Premarket Notification for New Dietary Ingredient for Haematococcus pluvialis algae, Report 65:1-104, 2000, at page 12).

Naturally derived astaxanthin exists mainly in the form of the 3S,3′S stereo isomer found in Haematococcus algae or the 3R,3R′, which is found mainly in Phaffia yeast. Synthetic astaxanthin has a more complex stereo isomeric profile due to the non stereo selectivity from the reaction conditions used in its manufacture. Haematococcus pluvialis also contains mono and diesterified astaxanthin as the predominant forms of astaxanthin, while Phaffia and synthetically produced astaxanthin substantially lack these esterifications.

Natural astaxanthin extracts contain astaxanthin in different isomeric forms, the so called and E and Z isomeric configurations. The following provides a summary of the ranges of astaxanthin isomers analyzed in algae preparations from different Haematococcus producers:

TABLE 1
E/Z ratios of Astaxanthin isomers in various
sources of Haematococcus algae

| | All - E | | |
|---|---|---|---|
| Source of algae | Astaxanthin | 9-Z Astaxanthin | 13-Z Astaxanthin |
| Aquasearch¹ | 1.30 | 0.10 | 0.20 |
| Aquasearch¹ | 1.90 | 0.30 | 0.30 |
| Aquasearch¹ | 2.10 | 0.40 | 0.40 |
| US Nutra - | 2.88 | 0.48 | 0.68 |
| also expressed as (% | (70%) | (12%) | (16%) |
| of total) | | | |

Figures expressed as % w/w of Total Astaxanthin.
¹Aquasearch, FDA 75-day Premarket Notification for New Dietary Ingredient for Haematococcus pluvialis algae. Report 65: 1-104, 2000.

Typically natural astaxanthin extract derived from Haematococcus pluvialis comprises astaxanthin stereoisomers as follows: (3S,3′S) 100%; (3S,3′R) and (3R,3′S) 0%; (3R,3′R) 0%, with the geometric isomer proportions, expressed as a percentage of the total astaxanthin, of about: E-astaxanthin 59% ; 9Z-astaxanthin 15%; 13Z-astxanthin 26%, and non-astaxanthin carotenoid levels of about: 0.3% β-carotene, 0.07% lutein, 0.3% canthaxanthin and 1.3% total other carotenoids.

In addition to the carotenoid content of a natural astaxanthin extract, the extract will also contain fatty acids. The levels and mixture of fatty acids in the extract generally reflect the levels of fatty acids found in the source material. By way of example, the following fatty acids are found in Haematococcus pluvialis and include the following acids: Lauric, Tridecanoic, Myristic, Pentadecanoic, Palmitic, cis-9-Palmitoleic, Heptadecanoic, cis-10-Heptadecenoic, Stearic, cis-9-Oleic and/or trans-9-Elaidic, cis-9,12-Linoleic and/or trans-9,12-Linolelaidic, Arachidic, alpha-Linolenic, cis-11-Eicosenoic, Linolenic, Heneicosanoic, cis-11,14-Eicosadienoic, Behenic, cis-8,11,14-Eicosatrienoic, cis-13-Erucic, cis-11,14,17-Eicosatrienoic, cis-5,8,11,14-Arachidonic, and cis-5,8,11,14,17-Eicosapentaenoic acids.

Thus, in certain embodiments, a natural astaxanthin-enriched extract in the form of an oleoresin will contain from about 1-30% total astaxanthin, for instance, at least about 6-15% astaxanthin, for instance, about 10% astaxanthin. The oleoresin also comprises a mixture of naturally occurring fatty acids from the source material. For instance, in embodiments where the natural astaxanthin extract is prepared from Haematococcus algae, such as by way of supercritical fluid CO₂ extraction, examples of the oleoresin will comprise (expressed as the approximate total percent of fatty acids present): Lauric (0.5-0.7), Tridecanoic (0.09-0.1), Myristic (0.51-0.52), Pentadecanoic (0.03), Palmitic (12.21-13.14), cis-9-Palmitoleic (0.24-0.32), Heptadecanoic ( 0.1-0.11), cis-10-Heptadecenoic (1.76-1.87), Stearic (0.77-0.79), cis-9-Oleic and/or trans-9-Elaidic (24.14-24.37), cis-9,12-Linoleic and/or trans-9,12-Linolelaidic (30.30-30.68), Arachidic (1.77-1.86), gamma-Linolenic (14.15-14.83), cis-11-Eicosenoic (0.25-0.26), Linolenic (0.18), Heneicosanoic (1.15-1.65), cis-11,14-Eicosadienoic (0.48-0.53), Behenic (0.06), cis-8,11,14-Eicosatrienoic (1.34-1.40), cis-13-Erucic (0.06-0.07), cis-11,14,17-Eicosatrienoic (8.37-8.81), cis-5,8,11,14-Arachidonic (0.12), and cis-5,8,11,14,17-Eicosapentaenoic acids (0.05-0.06).

Phaffia rhodozyma is a form of yeast that also contains astaxanthin. Compared to synthetic astaxanthin and Haematococcus derived astaxanthin, Phaffia-derived astaxanthin is different in that it contains predominately the 3R, 3R′ stereoisomeric form of astaxanthin (Andrews and Starr, Phytochemistry 15:1003-1007, 1976) and the astaxanthin is present largely in the unesterified form (97%) with an E to Z ratio of about 60:40. By way of example, Phaffia-derived astaxanthin enriched extract can be produced using the methods described in Lim et al. (Biochem. Eng. J., 11(2-3): 181-187, 2002).

Pharmaceutical agent or drug: A chemical compound or composition capable of inducing a desired therapeutic or prophylactic effect when properly administered to a subject.

Supercritical Fluid Extraction (SFE or SCFE): Supercritical fluids are highly compressed gases that combine properties of gases and liquids. Supercritical fluids (e.g., supercritical fluid carbon dioxide) can be used to extract compounds, such as lipophilic or volatile compounds, from samples. Supercritical fluids are inexpensive, contaminant free, less costly to dispose of safely than organic solvents, and have solvating powers similar to organic solvents, but with higher diffusivities, lower viscosity, and lower surface tension. The solvating power can be adjusted by changing the pressure or temperature of the extraction process, or by adding modifiers to the supercritical fluid.

A typical supercritical fluid extractor consists of a tank of the mobile phase, such CO₂, a pump to pressurize the gas, an oven containing the extraction vessel, a restrictor to maintain a high pressure in the extraction line, and a trapping vessel. Analytes are trapped by letting the solute-containing supercritical fluid decompress into an empty vessel, through a solvent, or onto a solid sorbent material.

Examples of extraction systems are dynamic, static, or combination modes. In a dynamic extraction system, the supercritical fluid continuously flows through the sample in the extraction vessel and out the restrictor to the trapping vessel. In static system, the supercritical fluid circulates in a loop containing the extraction vessel for some period of time before being released through the restrictor to the trapping vessel. In a combination system, a static extraction is performed for some period of time, followed by a dynamic extraction.

The use of supercritical fluid extraction to obtain natural compounds and complexes is well known in the art. See, for instance, Natural Extracts Using Supercritical Carbon Dioxide, by Mamata Mukhopadhyay (CRC Press LLC, Boca Raton, Fla., 2000, ISBN 0-8493-0819-4).

Therapeutically effective dose or amount: A quantity of a substance, such as an antioxidant, sufficient to achieve a desired effect in a subject being treated. The effective amount of a specific substance will be dependent on the subject being treated, the severity of the affliction, and the manner of administration of the substance.

The therapeutically effective amount of a substance, such as the therapeutically effective amount of an antioxidant, can be determined by various methods, including generating an empirical dose-response curve, predicting potency and efficacy of a congener by using quantitative structure activity relationships (QSAR) methods or molecular modeling, and other methods used in the pharmaceutical sciences. Since oxidative damage is generally cumulative, there is no minimum threshold level (or dose) with respect to efficacy. However, minimum doses for producing a detectable therapeutic or prophylactic effect for particular conditions can be established.

Unless otherwise explained, all technical and scientific terms used herein have the same meaning as commonly understood by one of ordinary skill in the art to which this invention belongs. The singular terms “a,” “an,” and “the” include plural referents unless context clearly indicates otherwise. Similarly, the word “or” is intended to include “and” unless the context clearly indicates otherwise. Hence “comprising A or B” means including A, or B, or A and B. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including explanations of terms, will control. In addition, the materials, methods, and examples are illustrative only and not intended to be limiting.

### III. ASTAXANTHIN PREVENTS DNA OXIDATION IN IMMUNE CELLS

Disclosed is a method of reducing DNA cellular damage in vivo by administering an oral dose of natural astaxanthin extract to a subject. The natural astaxanthin extract is preferably in mono- and di-ester form, which is known to exhibit greater stability and intestinal absorption in comparison with free astaxanthin. Surprisingly, when administered orally, the natural astaxanthin extract greatly reduces in vivo oxidative damage to the subjects' cells, especially cells of the immune system.

Reported herein is a study of the role of dietary astaxanthin on immunity and oxidative status in healthy adult humans. Female subjects with no history of major diseases received 0, 2, or 8 mg astaxanthin, in the form of a natural astaxanthin-enriched extract from H. pluvialis, (n=14) daily for 8 weeks in a double-blind, placebo controlled study. Blood was drawn on wk 0, 4 and 8. The tuberculin test was assessed on week 8. Plasma astaxanthin was undetectable prior to feeding but increased (P<0.01) dose-dependently on weeks 4 and 8. Dietary astaxanthin stimulated concanavalin A-, phytohemagglutinin- and pokeweed mitogen-induced lymphoproliferation and increased NK cell cytotoxic activity. In addition, astaxanthin increased the proportion of total T cells and B cells, but did not influence the populations of Th, Tc or NK cells or the ratio of Th:Tc cells. On week 8, the frequency of cells expressing LFA-1 marker was higher in subjects given 2 mg (42.1%) but not those given 8 mg (30.6%) astaxanthin as compared to control (31.8%). No similar dietary effect was observed with ICAM-1 or LFA-3 expression. Subjects fed 2 mg but not those fed 8 mg astaxanthin had higher DTH response than unsupplemented controls. Astaxanthin feeding did not influence lipid peroxidation in plasma.

Dietary astaxanthin dramatically decreased blood DNA damage (measured as the level of 8-OHdG) after 4 weeks of feeding. This is particularly surprising not only because of the magnitude of the effect (>35% reduction in 8-OHdG), but also in light of prior report indicating that oral carotenoid supplementation did not have a significant effect on endogenous oxidative DNA damage (Collins et al., Carcinogenesis 19(12):2159-2162, 1998).

Thus, there is provided herein a method for reducing (for instance, by preventing, reversing, inhibiting, or ameliorating) oxidative DNA damage in a subject, which method involves administering to the subject a therapeutically effective amount (or dose) of astaxanthin, particularly astaxanthin in the form of a natural extract. By way of example, representative and non-limiting methods of, or references teaching, extracting, purifying, and/or manufacturing astaxanthin preparations are described herein.

### IV. ASTAXANTHIN

Astaxanthin is well known as the pigment providing the pinkish-red hue to the flesh of salmon and trout, as well the coloring in the carapaces of shrimp, lobsters and crayfish. As animals are unable to synthesize carotenoids, these animals obtain astaxanthin through the food chain from the sources which manufacture it.

The structure of astaxanthin has been determined (Grangaud, Comt. Rend., 242, 1767, 1956; Andrews et al., Acta. Chem. Scand., B28, 730, 1974), and is as follows: 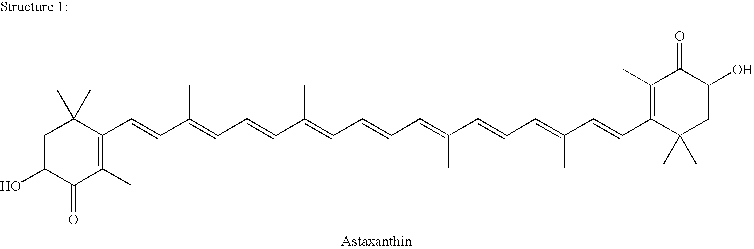
The International Union of Pure and Applied Chemistry (IUPAC) name for astaxanthin is (3,3′-dihydroxy-beta,beta-carotene-4,4′-dione).

The astaxanthin molecule has two asymmetric carbons located at the 3 and 3′ positions of the benzenoid rings on either end of the molecule. Different enantiomers of the molecule result from the exact way that the hydroxyl groups (—OH) are attached to the carbon atoms at these centers of asymmetry. When the hydroxyl group is attached so that it projects above the plane of the molecule, it is said to be in the R configuration; when the hydroxyl group projects below the plane of the molecule, it is said to be in the S configuration. Thus the three enantiomers of astaxanthin are designated (3R,3′R), (3S,3′S) and (3R,3′S; meso). These different enantiomeric forms are shown in the following structures:

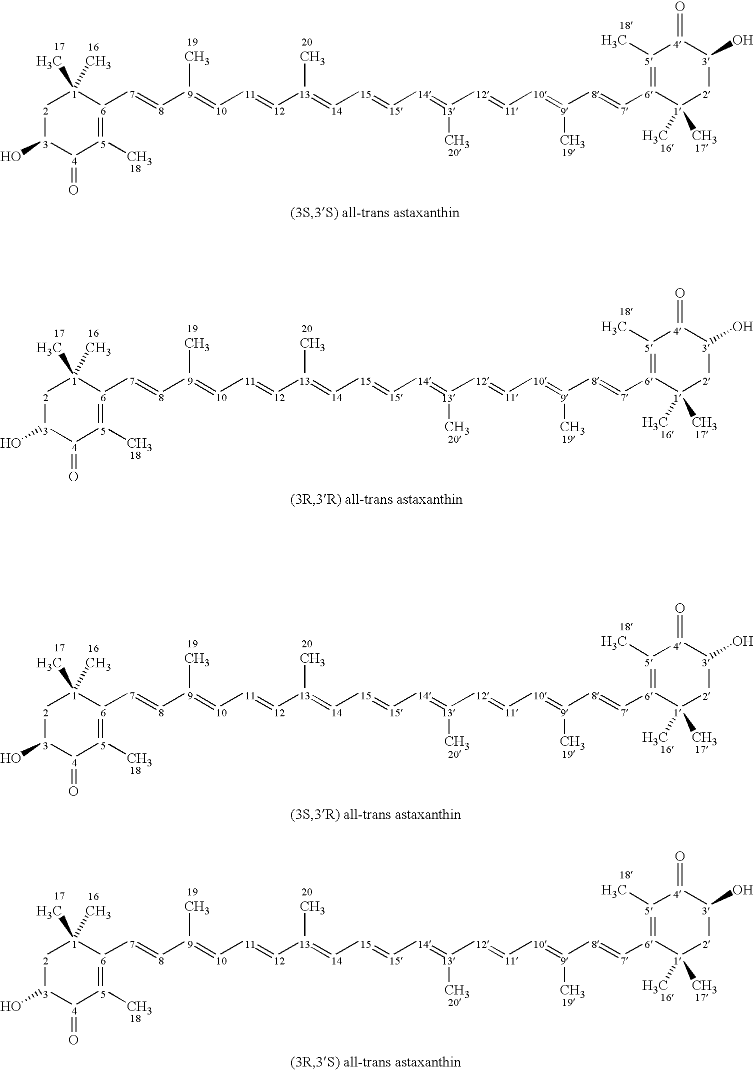

This cis-trans or (E/Z)-isomerism of the carbon-carbon double bonds is another interesting feature of the stereochemistry of carotenoids, such as astaxanthin, because it has been demonstrated that the (E/Z)-isomers may have different biological properties. According to the number of double bonds, a great number of hypothetical (E/Z)-isomers exist for each carotenoid. In view of the (E/Z)-isomerism, the double bonds of the polyene chain can be divided into two groups: (I) double bonds with no steric hindrance of the (Z)-isomer (central 15,15′-double bond and the double bonds bearing a methyl group, such as the 9-, 9′-, 13-, and 13′-double bonds) and (2) double bonds with steric hindrance (7-, 7′-, 11-, and 11′-double bonds). Although isomers with sterically hindered (Z)-double bonds are known, the number of possible (Z)-isomers is in reality reduced considerably due to steric hindrances.

Normally, carotenoids occur in nature as the (all-E)-isomer, though exceptions are known. Some carotenoids readily undergo isomerization when isolated or otherwise manipulated; therefore (Z)-isomers that are described in the literature as natural products may be artifacts. In addition, (E/Z)-isomerization may occur when a carotenoid is kept in solution. Normally, the percentage of the (Z)-isomers is rather low, but it is enhanced at higher temperature, and the formation of (Z)-isomers is increased by exposure to light.

Østerlie et al. (Abs. 2A-13; 12th Int. Symp on Carotenoids, Cairns, Queensland, AU, 1999) discusses the blood appearance and distribution of astaxanthin E/Z isomers amount plasma lipoproteins in humans administered a single meal of astaxanthin.

Synthetically produced astaxanthin is normally present in unesterified form (i.e., diol). In nature, astaxanthin is often present as diesters. It is known that astaxanthin present as diester is more stable than free astaxanthin (Omara-Alwala et al., J. Agric. Food Chem., 33:260, 1985; Arai et al., Aquaculture, 66:255, 1987). In addition, it is believed that esterified (mono- or diester, or a mixture thereof) astaxanthin is more biologically available/active.

Astaxanthin and/or its ester can be chemically synthesized by any method for use in the compositions and methods described herein. Methods for synthesizing astaxanthin are established (Cooper et al., J. Chem. Soc. Perkin Trans. I, 2195, 1975; Kienzle et al., Helv. Chim. Acta, 61, 2609, 1978; Widmer et al., Helv. Chim. Acta., 64, 2405, 1981; Mayer et al., Helv. Chim. Acta., 64, 2419, 1981); see also the disclosures in U.S. Pat. No. 5,654,488 to Krause et al., and U.S. Pat. No. 4,245,109 to Mayer et al. In addition, chemically synthesized astaxanthin products are readily available, for instance, from DSM Nutritional Products (Basel, Switzerland) (formerly Roche Vitamins and Fine Chemicals), Bayer's chemical division (sold as Carophyll® pink; Roche Vitamins Japan KK, Tokyo, Japan), and BASF (sold as Lucantin® Pink; Mount Olive, N.J.).

Although natural sources of astaxanthin are numerous, nearly all produce only very low concentrations. The green algae Haematococcus pluvialis provides the most concentrated natural source of astaxanthin known, from 10,000-40,000 ppm (mg/kg) astaxanthin. As a comparison, the flesh of wild Atlantic salmon on average contain 5 ppm of astaxanthin, Coho salmon about 14 ppm astaxanthin and sockeye salmon average 40 ppm (Turujman et al., J AOAC Int. 80(3):622-632, 1997). A typical gelcap comprising a 1 mg dose of astaxanthin from Haematococcus has the same amount of astaxanthin as 200 grams of Atlantic salmon. Astaxanthin and/or its ester, has been found in krill, in shrimp eggs (Kuhn et al. Angew. Chem. 51, 465, 1938), in animal organs (Kuhn et al., Ber., 72, 1688, 1939), in plants (Tischer et al., Z. Physiol. Chem., 267, 281, 1941), in the petals of Amur adonis and buttercups (Seybold et al., Nature, 184, 1714, 1959) and the red wings of birds (Z. Physiol. Chem., 288, 20, 1951).

Astaxanthin can be extracted and purified from natural sources for use in the methods and compositions described herein. For instance, astaxanthin can be isolated from Haematococcus algae. Haematococcus occurs in nature worldwide, but is most often found in cooler pools of fresh water. Under these conditions, Haematococcus is motile and utilizes the available nitrate, phosphate, and other nutrients to grow and reproduce. However, when nutrients become limiting or the pool begins to dry, the alga form a protective cell wall and encyst. Massive amounts of astaxanthin are produced, and the cells undergo a dormant stage until the next influx of water and nutrients. Cells can remain viable in this encysted stage with the high level of protective astaxanthin for decades. Red cysts are significantly more resistant to photoinhibition and oxygen radicals than green cells, suggesting significant protective roles for astaxanthin (Kobayashi et al., J. Ferm. Bioeng. 74(1):61-63, 1992).

U.S. Pat. No. 4,871,551 to Spencer, describes growth of Haematococcus cells and subsequent grinding to extract astaxanthin. U.S. Pat. No. 6,022,701 describes a method for obtaining a large amount of astaxanthin by inducing cyst formation in algae after aerobically culturing Haematococcus pluvialis. A method for increasing the formation of astaxanthin by Haematococcus pluvialis by adjusting the concentration ratio of carbon to nitrogen (C:N) in the culture is described in PCT Japanese National Publication No. 2-501189. In addition, Japanese Unexamined Patent Publication No. 5-68585 describes a method for obtaining a large amount of astaxanthin by inducing cyst formation in algae after aerobically culturing Haematococcus pluvialis. Japanese Unexamined Patent Publication No. 1-187082 describes methods for producing astaxanthin and/or its ester by culturing green algae able to biosynthesize astaxanthin, examples of which include Clamvdomonas, Haematococcus, Chlorocytrium, Chlorella, Chlorococcum, Characium, Trebouxia, Dictyosphaerium, Scenedesmus, and Hydrodictycm, in a medium containing sodium, potassium and rubidium salts. See also U.S. Pat. No. 5,607,839 to Tsubokura et al. and U.S. Pat. No. 5,811,273 to Misawa et al.

Animal studies have proven the safety of consuming Haematococcus algae. It has never been associated with any toxicity in the reported literature or in published field studies. Haematococcus algae has been reviewed by the US FDA and approved as a dietary supplement. It has also been approved in Japan for use in both foods and animal feeds. A different formulation of Haematococcus algae extract has gained wide acceptance in the aquiculture markets as a pigmentation and vitamin source for salmon, trout, shrimp and ornamental fish and has been approved as a feed additive for salmonids in Canada.

Standard toxicity and safety studies have been conducted with Haematococcus algae. Acute oral toxicity studies were conducted on Charles River CD rats with a dosage level of 5 grams of Haematococcus algae/kg for 13 days. Groups were evaluated for mortality, pharmacotoxic signs, body weights, and necropsy examinations during the 13-day study. The demonstrated LD₅₀ value of each lot was greater than the administered dose of 5 grams/kg. No visible abnormalities were observed, nor differences in body weights during the study. Postmortem examination did not reveal any abnormalities in rats sacrificed at the end of the study. A second clinical acute toxicity study with rats showed a LD₅₀ value higher than 12 grams/kg with no clinical, weight or behavioral abnormalities. Postmortem pathology showed no appreciable macroscopic findings at the end of the 14 days; in addition, hematology, blood chemistry, urinalysis, organ weight, and gross pathology were all clinically normal.

Higher dosage studies of acute oral toxicity have been conducted with both male and female mice ranging from 10.4-18.0 grams Haematococcus algae per kg of body weight with no mortalities or abnormalities observed at the end of the study. Mutagenicity tests under standard conditions are negative for Haematococcus algae. Another published study with rats fed 400 ppm astaxanthin for 41 days showed no harmful effects on body/organ weight, enzyme activities, pregnancy, or litter size (Nishikawa et al., Koshien Daigaku Kiyo 25:19-25, 1997).

The safety of astaxanthin-enriched Haematococcus pluvialis extract has also recently been demonstrated in humans (J. Med. Food, 6(1):51-56, 2003). Thus, there is every indication that Haematococcus algae extract is a safe and natural form of astaxanthin that has been shown to have excellent antioxidant properties.

By way of example, one Haematococcus algae extract that is useful in the current methods is ZANTHIN® Extract Astaxanthin Complex 10% Standardized (U.S. Nutra, LLC, Eustis, Fla.). The following is a representative chemical analysis of a batch of this extract: Astaxanthin Complex>10% (Quantified spectrophotometrically against standard [Sigma A9335] in acetone (λmax 478)), containing: Astaxanthin>9.5%; Lutein˜0.1%; β-Carotene˜0.1%; Canthaxanthin˜0.1%; and Other Carotenoids˜0.2%. The ZANTHIN® Extract Astaxanthin Complex is prepared using a supercritical fluid (CO₂) extraction process (SuPure® CO₂) that produces a product containing no solvent residues.

Another contemplated preparation comprising astaxanthin that is appropriate for use in methods described herein is astaxanthin in the form of an oleoresin concentrate from Haematococcus pluvialis, marketed as astaZanthin™ by La Haye Laboratories Inc., Redmond, Wash. The ratios of components of this extract are believed to be similar to those listed above for ZANTHIN®.

Astaxanthin also can be extracted from Adonis species plants (see, e.g. as disclosed in U.S. Pat. No. 5,453,565 to Mawson, and Japanese Published Patent Publication No. 5-509227), and from yeast (see, e.g., U.S. Pat. Nos. 5,346,810 and 5,972,642 to Fleno et al., Japanese Unexamined Patent Publication No. 3-206880, and Japanese Unexamined Patent Publication No. 4-228064). Methods for extracting astaxanthin and/or its ester from the shells of crustacean are described in U.S. Pat. No. 4,505,936 to Meyers et al. and Japanese Unexamined Patent Publication No. 58-88353.

Additional methods and refinements for extracting and/or purifying astaxanthin are described in the following: U.S. Pat. No. 6,743,953 to Kumar et al.; U.S. Pat. No. 6,365,386 to Hoshino et al.; U.S. Pat. No. 5,210,186 to Mikalsen et al.; Japanese Unexamined Patent Publication No. 60-4558; Japanese Unexamined Patent Publication No. 61-281159; Japanese Unexamined Patent Publication No. 5-155736; and Yamashita (Food and Development, 27(3): 38-40, 1992).

It is believed that any astaxanthin preparation, or preparation of its esters, or natural extract comprising astaxanthin, can be used in the disclosed methods and compositions. In some embodiments, it is beneficial to use a natural astaxanthin-enriched extract, such as an extract prepared from H. pluvialis, in the described methods. Synthetic astaxanthin, as discussed above, can be produced by various chemical methods and the synthetic processes result in a mixture of different stereoisomers. Biosynthesized astaxanthin as produced a number of different organisms also result in varying stereoisomeric/enantiomeric forms. These differences are highlighted in Table 2.

TABLE 2
Enantiomers Found in Astaxanthin from Various Sources

| | | (3S,3′R) | |
|---|---|---|---|
| SPECIES | (3S,3′S) | and (3R,3′S) | (3R,3′R) |
| Yeast (Phaffla sp.) | — | <2% | >98% |
| Micro algae (Haematococcus) | 100% | — | — |
| Synthetic Astaxanthin | 25% | 50% | 25% |
| (Carophyll Pink ™, La Roche) | | | |
| Atlantic Salmon | 78-85% | 2-6% | 12-17%¹ |

¹(Schiedt et al., Helv Chim. Acta 64, 449-457, 1981)

Another difference between synthetic and natural (e.g., Haematococcus derived) form astaxanthin is that the naturally derived material mainly consists of mono and diesterified astaxanthin fatty acid esters. These are also the predominant form found in salmon species, and appear to be more bio-available (possibly because it is better absorbed). Many biological studies have been conducted on the different forms of Astaxanthin and studies by Naguib have shown that the Haematococcus astaxanthin containing extract is more potent anti oxidant in vitro than for example the synthetic form (Naguib, J. Agric. Food Chem, 48:1150-1154, 2000). Synthetic astaxanthin is also less stable to oxidative degradation, which reduces is effective shelf life unless it is stored under vacuum and or frozen.

Thus, natural astaxanthin extract has several advantages. First, almost all of the material extracted from H. pluvialis is in the 3S, 3S′ configuration, the identical isomeric form found in primarily nature, for instance, in salmon. Most of the experimental data on natural astaxanthin related to biological effectiveness has used this isomer. The Phaffia astaxanthin on the other hand is all 3R,3R′. While found in nature, this form is a tiny fraction of the total found or produced by any organism. Few organisms utilize the 3R, 3R′ form, but it has proven ability to color Atlantic salmon when used as a feed.

Astaxanthin extracted from Haematococcus algae is primarily in the esterified form, both monoester and diester forms. Esters are chemically more stable than free astaxanthin. Phaffia yeast astaxanthin is non-esterified (all free, diol). Synthetic astaxanthin is comprised of all free, non-esterfied astaxanthin in all four possible chiral forms (a racemic mixture). The racemic mixture is comprised of four forms, 25% 3R,3R′, 25% 3S,3S′ and 50% in the meso, (3R,3S′ & 3S,3R′) forms. Thus, only about 25% of synthetic astaxanthin is in the same form found naturally in salmon. In fact, analysis of astaxanthin in for the different chiral forms is the primary way to tell if a fish is farmed or wild (e.g., for the prevention of mislabeled product). Natural extracted astaxanthin-enriched oleoresin, such as that produced by U.S. Nutra, is in the most stable form of all, where 10% astaxanthin is dispersed in a solution of natural algal oil, comprised primarily of omega-3 and omega-6 fatty acids.

By way of example, natural astaxanthin enriched extracts useful in the provided methods comprise predominantly esterified astaxanthin. For instance, an example of such an extract from Haematococcus species will contain the various forms of astaxanthin and other carotenoids in the following amounts (based on total astaxanthin present of between 81-99%): free astaxanthin 1-5%; astaxanthin monoesters 46-79%; astaxanthin diesters 10-39%; β-carotene 0-5%; lutein 1-11%; canthaxanthin 0-5.5%; and other carotenoids 1-9%. Specific extracts will contain free astaxanthin at about 0.20-0.73%; astaxanthin monoesters at about 80-82%; and astaxanthin diesters at about 14.80-16.60%.

An extract containing astaxanthin and/or its ester obtained by any of these methods, or equivalent methods or other methods known to those of ordinary skill in the art, can be used in the described methods for inhibiting DNA damage. For instance, also useful in the provided methods are relatively crude extracts or powders containing astaxanthin and/or its ester, which extract/powder has been suitably purified as necessary. It is particularly contemplated that the astaxanthin-containing extract in some embodiments will contain additional naturally-occurring carotenoids, which collection of total carotenoids in the extract can be referred to as a carotenoid complex or, more specifically (where astaxanthin is the predominant carotenoid in the complex), an astaxanthin complex of carotenoids.

In all embodiments, it is contemplated that the astaxanthin preparation can be provided to the subject alone or in a formulation with one or more additional components.

One possible, non-limiting, mechanism of action of astaxanthin is through its antioxidant activity. Through this antioxidant action, astaxanthin may be involved in aging, cardiovascular diseases, dermatology disorders, cancer, immune function, inflammation, gastrointestinal diseases, strength and endurance, ocular diseases (macular degeneration), and neurological (Parkinson's and Alzheimer's) diseases. Overproduction of reactive oxygen and nitrogen species can tip the oxidant:antioxidant balance, resulting in the various diseases mentioned. Therefore, dietary antioxidants are needed to remove these harmful oxidative products that can destroy cell membranes, proteins and DNA. Another characteristic of astaxanthin as a dietary carotenoid is its absorption rate. It has been shown, for instance, that the concentration of astaxanthin in plasma was much higher than that of β-carotene lutein in mice fed the same amount of these carotenoids (Park et al., J. Nutr. 128: 1802-1806, 1998; Park et al., J. Nutr. 128:1650-1656, 1998).

### V. DETECTION AND QUANTIFICATION OF OXIDATIVE DNA DAMAGE

Oxidative DNA damage can be measured by any art known technique. Methods for assessing DNA damage are well known; see, for instance, Loft & Poulsen (Free Radic. Res. 33:S67-83, 2000). By way of example, the level of oxidative DNA damage in an organ or cell may be studied by measurement of modified bases in extracted DNA by immunohistochemical visualization, and from assays of strand breakage before and after treatment. Oxidatively modified nucleobases can be measured in the DNA and strand breaks can be detected by the comet assay, optionally with the use of repair enzymes introducing breaks at oxidized bases. Oxidized bases and nucleosides from DNA repair, the nucleotide pool and cell turnover can be measured in urine. The excretion rate represents the average rate of damage in the body, whereas the level of oxidized bases in DNA is a concentration measurement in the specific cells.

The comet assay, also called the ‘Single Cell Gel Assay’, is a well known technique to detect DNA damage and repair at the level of single cells. This technique was developed by Swedish researchers Östling & Johansson (Biochem. Biophys. Res. Commun. 123:291-298, 1984), who demonstrated that DNA in one or a few cells embedded in low-melt agarose migrates out of the cell in an electrophoretic field in a pattern that is influenced by the extent of the DNA damage. The comet assay was later modified by Singh et al. (Exp. Cell Res., 175:184-191, 1988), and is now described as the alkaline comet assay. The comet assay is one of the most popular tests of DNA damage (e.g., single- and double-strand breaks, oxidative-induced base damage, and DNA-DNA/DNA-protein cross linking) detection by electrophoresis that has been developed. The assay is described and reviewed in the following references: McKelvey-Martin et al., Mutat. Res. 288: 47-63, 1993; Fairbairn et al., Mutat. Res. 339: 37-59, 1995; Anderson et al., Mutagenesis 13: 539-555, 1998; Rojas et al., J. Chromat. B Biomed Sci Appl 722: 225-254, 1999; Tice et al., Environ Mol Mutagen 35(3):206-21, 2000; Collins, Methods Mol. Biol. 203:163-177, 2002; Olive, Methods Mol. Biol. 203:179-194, 2002; Faust et al., Mutat. Res. 566:209-229, 2004.

In addition, the comet assay can be adapted in order to detect oxidized pyrimidines and purines (such as 8-oxo-guanine) by digestion of the embedded nucleoid samples with endonuclease III and formamidopyrmidine glycosylase (FPG), respectively. The additional breaks formed at the site of base oxidations increase the relative amount of DNA in the tail of the resultant comet. See, for instance, Collins et al., Carcinogenesis 19:2159-2162, 1998.

8-Hydroxy-2′-deoxyguanosine (8-OHdG) is one of the most commonly used markers for assessing oxidative DNA damage. This compound is also sometimes referred to as 8-oxy-7-hydrodeoxyguanosine (8-oxodG). DNA can be oxidized to produce many oxidative products; however oxidation of the C-8 of guanine is one of the more common oxidative events, and results in a mutagenic lesion that produces predominantly G-to-T transversion mutations. 8-OHdG can be measured in DNA samples (such as lymphocyte DNA) and in urine (Wu et al., Clin. Chim. Acta. 39:1-9, 2004). Several methods for quantitating this biomarker are available. HPLC with electrochemical detection (HPLC/ECD) and GC/MS methods are widely used (see, e.g., Cadet et al., Free Radic. Biol. Med. 33:441-49, 2002; Cooke et al., Free Radic. Res. 32:381-397, 2000). Enzyme-linked immunosorbent assay (ELISA) techniques are also being employed (Santella, Canc. Epidemiol. Biomarkers Prev. 8:733-739, 1999).

Additional methods of assaying and/or quantifying oxidative damage to DNA are known to those of ordinary skill in the art. See, for instance, Cadet et al., Biol. Chem. 383:933-943, 2002; Kasai, Free Radic. Biol. Med. 33:450-456, 2002; and Halliwell, Am. J. Clin. Nutr. 72:1082-1087, 2000.

As used herein, a reduction in oxidative DNA damage is any measurable reduction in oxidized DNA in a subject, or any measurable reduction in a marker for oxidized DNA. Thus, for instance, a reduction in oxidation DNA damage can be measured as reduction in the size of comet observed, using a comet assay, or a reduction in the level of an oxidative DNA product (such as 8-OHdG) in a subject, compared to a time before administration of the astaxanthin composition, or in comparison to a subject not receiving the astaxanthin composition. In certain embodiments, the reduction is a reduction in the endogenous level of oxidative DNA damage.

By way of example, methods provided herein will result in at least a 10% reduction in oxidative DNA damage. In other embodiments, administration of the astaxanthin or astaxanthin-enriched extract results in at least a 15% reduction in oxidative DNA damage; at least 25% reduction, at least 30% reduction, at least 40% reduction, or more. In particularly beneficial embodiments, the level of endogenous oxidative DNA damage is reduced by at least 20% or more, for instance, at least 25%, at least 30%, at least 40%, at least 50%, at least 60%, at least 75%, at least 80%, or more. The reduction in oxidative DNA damage may be transient, and is expected to be linked to the dosage and time (duration) of administration of the astaxanthin or astaxanthin-enriched extract.

It is understood that a measured reduction in oxidative DNA damage may include outright prevention of the oxidative damage, reversal of damage that has already occurred, or a combination of these.

### VI. METHODS OF USE AND FORMULATION OF COMPOSITIONS

The present disclosure includes a treatment or supplement that inhibits DNA oxidation in a subject such as an animal, for example a rat or human. The method includes administering astaxanthin (pure or in the form of an extract), or a combination of astaxanthin and one or more other pharmaceutical or nutritional agents, to the subject optionally in a pharmaceutically compatible carrier. The astaxanthin is administered in an effective amount to measurably reduce, prevent, inhibit, reverse or otherwise decrease oxidative DNA damage in a cell of the subject, for instance an immune cell.

The treatment can be used prophylactically in any subject, since all subjects are exposed to oxidative damage through metabolic processes. In addition, the treatment can be supplied to a subject in a demographic group at significant risk for particular oxidative damage. Subjects can also be selected using more specific criteria, such as a definitive diagnosis of a condition leaving the subject prone to the depredations of oxidative DNA damage. The administration of any exogenous astaxanthin would inhibit the progression of the oxidation associated disease as compared to a subject to whom the astaxanthin was not administered. The antioxidant effect, however, increases with the dose of astaxanthin.

The vehicle in which the astaxanthin is delivered can include pharmaceutically acceptable compositions of astaxanthin using methods well known to those with skill in the art. Any of the common carriers, such as sterile saline or glucose solution, can be utilized with the drugs provided by the invention. Routes of administration include but are not limited to oral, intracranial ventricular (icv), intrathecal (it), intravenous (iv), parenteral, rectal, topical ophthalmic, subconjunctival, nasal, aural, sub-lingual (under the tongue) and transdermal. The astaxanthin may be administered intravenously in any conventional medium for intravenous injection such as an aqueous saline medium, or in blood plasma medium. Such medium may also contain conventional pharmaceutical adjunct materials such as, for example, pharmaceutically acceptable salts to adjust the osmotic pressure, lipid carriers such as cyclodextrins, proteins such as serum albumin, hydrophilic agents such as methyl cellulose, detergents, buffers, preservatives and the like. For instance, U.S. Pat. No. 6,132,790 to Schlipalius describes methods of making water miscible compositions comprising carotenoids, such as astaxanthin.

Astaxanthin and/or its crude extract can be used directly after being dissolved in ethanol and diluted with water. It can also be prepared into a latex preparation. A latex preparation can be prepared by adding gallic acid, L-ascorbic acid (or its ester or salt), gum (e.g., locust bean gum, qua gum or gelatin), vitamin P (e.g., flavoids such as hesperidin, lutin, quercetine, catechin, thianidine and eliodictin or mixtures thereof) to the aqueous phase, or by adding astaxanthin, astaxanthin crude extract or a mixture thereof to the oil phase, and then adding glycerine fatty acid ester or oil, examples of which include vegetable seed oil, soy bean oil, corn oil and other routinely used liquid oils. A high-speed agitator or homogenizer can be used to emulsify such compositions.

Astaxanthin and/or its ester is substantially insoluble in water. It can be provided in capsules and the like, for instance by suspending the astaxanthin in oil directly or by way of incorporation with an emulsifier. Alternatively, the astaxanthin product can be used in a powder, for instance, it can be spray dried and provided in the form of a liquid or powder. By way of example, U.S. Pat. Nos. 6,976,575 and 5,827,539, both to Gellenbeck, describe production of dry carotenoid-oil powders. Since the solubility of astaxanthin in oil is extremely low, although considerable time is required to dissolve crystals of astaxanthin in oil, the dissolution rate can be increased by using fine crystals. The solubility of astaxanthin is greater when heated to about 100° C. or above.

Esters of astaxanthin are highly soluble in, and can be easily dissolved in, oils. Examples of such oils include vegetable oils such as soy bean oil, corn oil, rape seed oil, palm oil, olive oil, safflower oil, lemon oil, orange oil, peanut oil and sunflower oil, hardened oils produced by hydrogenating these oils, natural waxes such as lanolin, whale wax and bees wax, animal fats such as beef tallow, pork tallow and butter as well as wheat germ oil and concentrated vitamin E oil. In addition, glycerine fatty acid ester, sucrose fatty acid ester, sorbitan fatty acid ester, soy bean phospholipid, propylene glycol fatty acid ester and stearate diglyceride can be used as emulsifiers.

Embodiments of the disclosure comprising compositions, including food and pharmaceutical compositions, that can be prepared with optional conventional acceptable carriers, adjuvants and/or counterions as would be known to those of ordinary skill in the art. Suitable excipients include, e.g., organic and inorganic substances that are appropriate for enteral, parenteral, or oral administration, e.g., water, saline, buffers, vegetable oils, mineral oils, benzyl alcohol, cyclodextrin, hydroxypropylcyclodextrin (for instance, beta-hydroxypropylcyclodextrin), polyethylene glycols, glycerol triacetate and other fatty acid glycerides, gelatin, soya lecithin, carbohydrates such as lactose or starch or other sugars, magnesium stearate, talc or cellulose. The preparations can be sterilized and/or contain additives, such as preservatives or stabilizers. Astaxanthin can be formulated with various oils, including coconut, sunflower, mustard, almond, sesame, safflower, or peanut.

For instance, for use in the provided methods an compositions, astaxanthin (in pure form or in the form of an extract) can be mixed in an oil, then encapsulated in softgel capsules for oral ingestion. The oils can vary and in various embodiments include virtually any edible or consumable oil, particularly vegetable oils including but not limited to natural oils, such as omega-3 and omega-6 fatty acids found in the Haematococcus algae, rice bran oil, olive oil, cranberry seed oil, or mixtures of two or more thereof.

The compositions in some embodiments are in the form of a unit dose in solid, semi-solid and liquid dosage forms such as tablets, pills (such as enteric-coated pills), capsules, powders, stabilized beadlets (which optionally are compressed into a tablet or other form), granules, suppositories, liquid solutions or suspensions, injectable and infusible solutions.

Although the dose varies according to the purpose of administration and status of the patient (sex, age, body weight and so forth), the normal adult dose as astaxanthin in the case of oral administration is 0.1 mg (100 μg) to 10 g per day and preferably 0.1 mg (100 μg) to 1 g per day. The range for obtaining preventive effects is 0.01 mg (10 μg) to 100 mg per day, for instance about 0.1 mg (100 μg) to 10 mg per day. Specific example daily dosages include 500 μg, 1 mg, 2 mg, 3 mg, 4, mg, 6 mg, 8 mg, 10 mg, and so forth, for instance to be provided to an adult human.

Alternatively, dosages in some embodiments are applied in order to raise the plasma astaxanthin in the subject above a steady state level for a period of time, for instance, for a period of at least one week, or more. Steady state astaxanthin in many subjects is often essentially undetectable when measured by HPLC. Thus, in various embodiments, dosages of astaxanthin are administered to a subject to increase the plasma astaxanthin level to at least 0.05 μmol/L (μmolar, or μM). In other embodiments, the level is increased to at least 0.06 μM, at least 0.08 μM, at least 0.1 μM, at least 1.2 μM, at least 1.4 μM or more. In various embodiments, the level of astaxanthin is maintained for more than a week, for instance, for at least two weeks, at least a month, or longer. In some instances, it is beneficial to continue maintenance of the astaxanthin dosage, and therefore the level of astaxanthin in the subject's system, for periods measured in months or years.

In carrying out the methods provided herein, there may be used a compound (such as astaxanthin) as defined in its free form or in the form of an ester, or in a mixture of free and esterified form(s). Typically such esters are C₁ to C₁₈ esters, such as ethyl esters, or esters with long chain fatty acids, such as lauric, myristic or palmitic esters, or naturally occurring esters. All forms can be provided to a subject individually or a mixture of forms obtained from natural products or compositions synthetically produced.

The preparations and methods described herein can be utilized in both human and veterinary medicine.

In another aspect, the disclosure provides a food supplement or pharmaceutical composition, which composition comprises astaxanthin or an ester thereof together with a food supplement or pharmaceutically accepted diluent or carrier.

In carrying out the methods provided herein, the astaxanthin may be used together with other active agents, such as, for example: another carotenoid (e.g., lycopene or alpha, beta, gamma or delta carotene), one or more other antioxidants (such as vitamin A, vitamin C, vitamin E (α-tocopherol and other active tocopherols)), selenium, copper, zinc, manganese and/or ubiquinone (coenzyme Q10). It is appreciated in the art that oral astaxanthin can be partially destroyed in the gastrointestinal tract, thereby lowering the effectively applied dosage. By providing vitamin E and/or vitamin C to the subject, this process in inhibited and more carotenoid is absorbed by the subject. The inhibitor may be included as part of a composition as part of a composition described herein, or administered separately.

The following examples are provided to illustrate certain particular features and/or embodiments. These examples should not be construed to limit the invention to the particular features or embodiments described.

### EXAMPLE 1

### Immune Stimulating Action of Dietary Astaxanthin

This example provides a description of effects of oral astaxanthin on the immune system of human adults, when taken at 2 mg or 8 mg per day.

Methods and Results

Free-living healthy female Korean subjects (average age 21.5 years) were recruited at Inha University, Korea. A three-day dietary record was obtained prior to the study to provide background dietary information. Subjects had no history of diabetes, cancer or alcohol abuse, and were non-smokers. They were allowed to consume their normal diets but advised to restrain from eating astaxanthin-rich foods. Subjects were assigned to receive 0 (control), 2, or 8 mg astaxanthin (109 g astaxanthin/kg oleoresin concentrate from Haematococcus pluvialis, astaZanthin™, La Haye Laboratories Inc., Redmond, Wash.) (n=14 subjects/diet) for eight weeks in a double-blind placebo control study. The astaxanthin was administered in the form of one soft gel capsule taken every morning. Blood was again drawn on week 0, 4 and 8 to assess immune function and oxidative status.

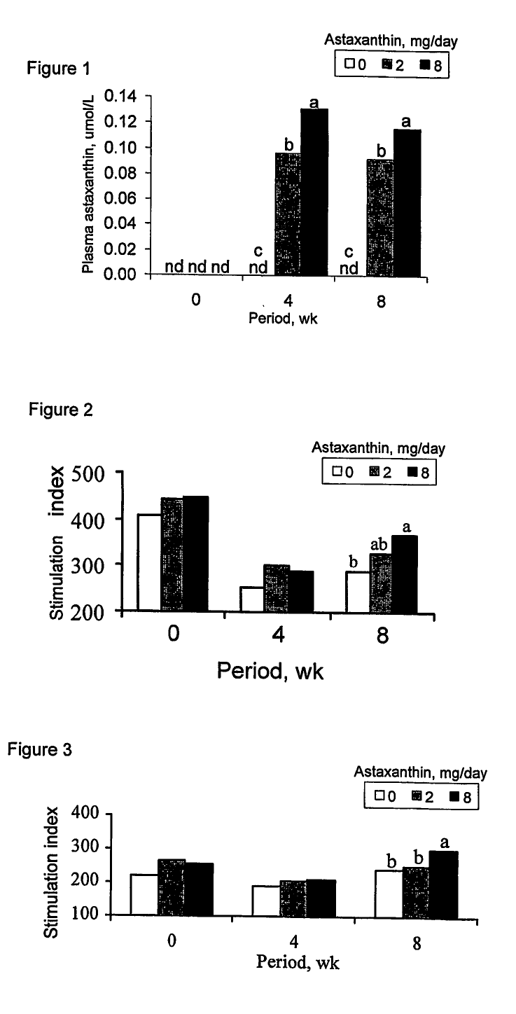

HPLC. Astaxanthin content in plasma was analyzed by reverse phase HPLC (Alliance 2690 Waters HPLC system fitted with a photodiode array detector, Waters, Milford, Mass.) as previously described (Park et al., J Nutr. 128(10):1650-1656, 1998). See FIG. 1 and Table 3. Trans-β-apo-8′carotenal (Sigma Chem. Co., St. Louis, Mo.) was used as the internal standard.

TABLE 3
Astaxanthin Levels

| | 0 mg | 2 mg | 8 mg |
|---|---|---|---|
| 0 week | nd | nd | nd |
| 4 week | nd | 0.0960 | 0.1309 |
| 8 week | nd | 0.0921 | 0.1162 |

nd = not detectable

Delayed-type hypersensitivity. Delayed-type hypersensitivity (DTH) response to an intracutaneous injection of tuberculin (Mono-Vacc Test (O.T.), Pasteur Merieux Connaught, France) was assessed on week 8. The injection was administered by a physician and skin thickness and induration were measured at 0, 24, 48 and 72 hours after challenge. In general, DTH response was maximal at 48 to 72 h post-injection (FIG. 9). Subjects fed 2 mg astaxanthin had higher (P<0.08) DTH response than unsupplemented controls. Those fed 8 mg astaxanthin did not show a similar heightened DTH response.

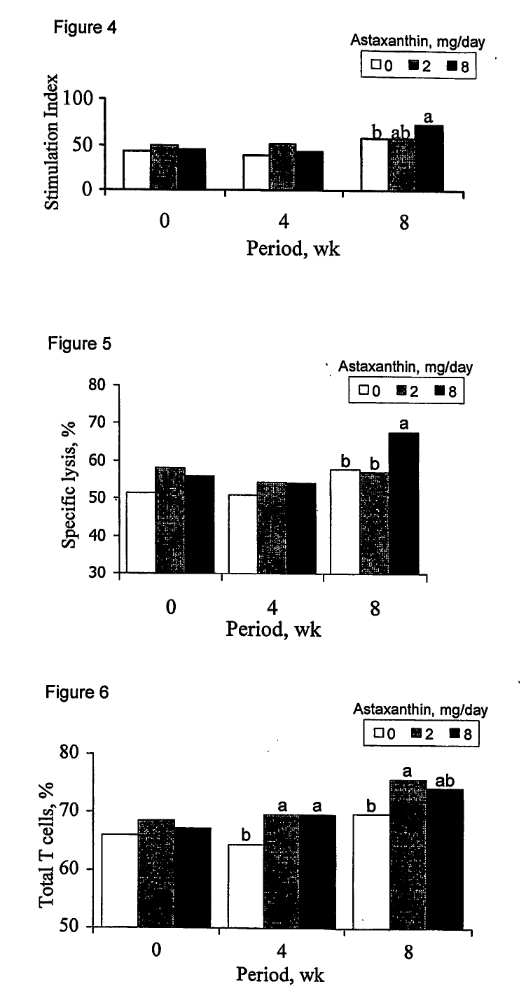

Lymphoproliferation. The proliferation response of peripheral blood mononuclear cell (PBMC) to PHA (2 and 10 mg/L final concentration), concanavalin A (Con A; 2 and 10 mg/L), and pokeweed mitogen (PWM; 1 and 5 mg/L) was assessed using whole blood cultures as described (Chew et al., J Nutr. 130(8):1910-1913, 2000). Whole blood was cultured in order to mimic in vivo conditions. Results were calculated as stimulation index (cpm of mitogen-stimulated culture/cpm of unstimulated cultures). Astaxanthin supplementation, especially those given 8 mg astaxanthin, increased lymphocyte proliferation when stimulated by the T cell-dependent mitogens PHA (FIG. 2) and Con A (FIG. 3), and also B cell mitogen (PWM, FIG. 4). The increases were significant (P<0.05) on wk 8 for all mitogens.

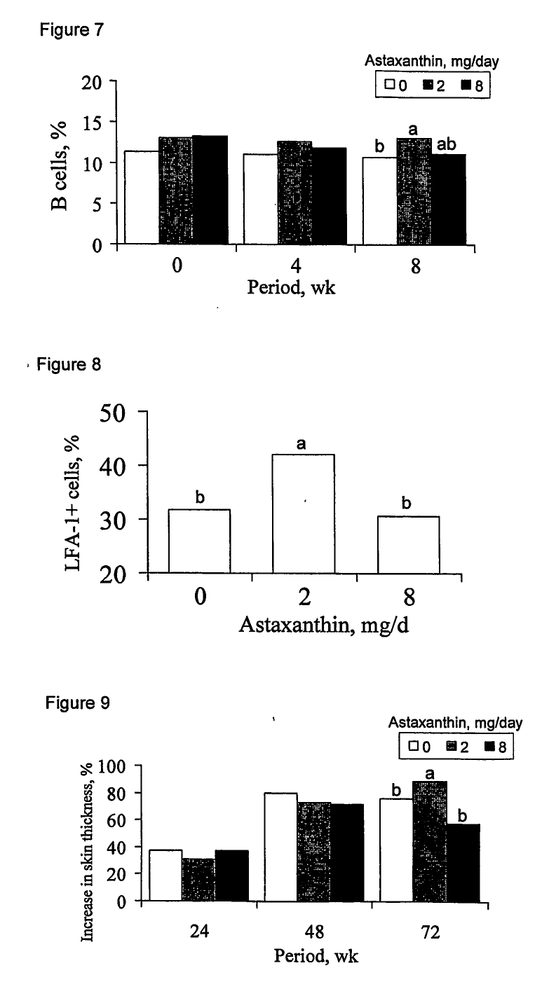

Leukocyte subset. Subpopulations of CD3 (total T), CD4 (Th), CD8 (Tc), NK (natural killer), and CD21 (B cells) were quantitated by flow cytometry as previously described (Chew et al. 2000). In addition, the distribution of the cell surface adhesion molecules ICAM-1 (CD54), LFA-1 (CD11a) and LFA-3 (CD58), also was measured by flow cytometry. The population of total T cells was higher (P<0.05) in astaxanthin-fed (both levels) subjects than unsupplemented controls on wk 4 and 8 (FIG. 6). The population of B cells also was higher (P<0.05) in subjects given 2 mg astaxanthin after 8 weeks (FIG. 7). On the other hand, higher (8 mg) dietary astaxanthin amounts did not elevate B cell population. Dietary astaxanthin did not significantly influence the population of Th, Tc or NK cells or the ratio of Th:Tc cells. The expression of LFA-1 (FIG. 8) but not LFA-2 adhesion molecules was increased (P<0.05) in subjects given 2 mg astaxanthin.

Natural killer cell cytotoxic activity. Effector (PBMC) and target (K562) cells were cultured at effector:target ratios of 5:1 and 10:1 in Dulbecco's Modified Eagles Medium (Sigma, St. Louis, Mo.) containing 100 mL/L fetal bovine serum, 100 U/mL penicillin, and 100 g/L streptomycin sulfate. Killing was assessed using MTT. Percent of specific cytotoxicity calculated as follows:
% Specific cytotoxicity=1−(OD_{effector+target}−OD_{effector})/OD_{target}×100.
Subjects given 8 mg astaxanthin had higher (P<0.05) NK cell cytotoxic activity by wk 8 of supplementation when the effector:target ratio was 10:1 (FIG. 5).

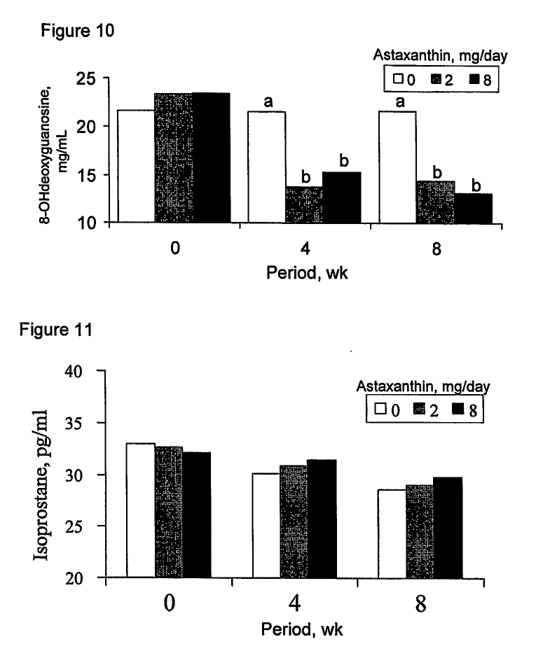

Oxidative damage to DNA. 8-Hydroxy-2′-deoxyguanosine (8-OHdG) was measured in plasma by ELISA (BIOXYTECH™ 8-OHhdG-EIA Kit, OxisResearch, Portland, Oreg.; sensitivity=0.5 μg/L). Subjects fed either 2 or 8 mg astaxanthin had dramatically lower (P <0.01) concentrations of 8-oxodG than unsupplemented subjects as early as wk 4 of feeding (FIG. 10 and Table 4). Higher dietary astaxanthin dose (8 mg) did not decrease further the DNA damage.

TABLE 4
Average Levels of 8-OHdG

| | 0 mg | 2 mg | 8 mg |
|---|---|---|---|
| 0 week | 21.6 | 23.4 | 23.5 |
| 4 week | 21.5 | 13.8 | 15.3 |
| 8 week | 21.7 | 14.4 | 13.2 |

Lipid-peroxidation. Plasma concentrations of 8-epi-prostaglandin F2α (8-isoprostane) was measured by ELISA. Astaxanthin did not significantly influence lipid peroxidation measured in the plasma (FIG. 11).

Statistics. Data were analyzed by repeated measures ANOVA using the General Linear Model of SAS (1991). Differences among treatment means were compared by a protected LSD test and considered different at P<0.05.

Discussion

Dietary astaxanthin enhanced both cell-mediated and humoral immune responses in healthy human subjects. The immune markers significantly enhanced by feeding astaxanthin included T and B cell mitogen-induced lymphoproliferation and NK cell cytotoxic activity. Enhancement of these ex vivo immune markers was supported by the observed increases in the total number of T and B cells as analyzed by flow cytometry. Similarly, the tuberculin DTH test (a reliable clinical test to assess in vivo T cell function; Miyamoto et al., J. Vet. Med. Sci. 57: 347-349, 1995) also was elevated in subjects given 2 mg astaxanthin. All these immune responses were generally observed after 8 weeks of supplementation, after cutaneous tuberculin injection. The heightened DTH response with dietary astaxanthin observed in the present study is in agreement with studies using β-carotene (Chew et al., J Nutr. 130(8):1910-1913, 2000) and lutein (Kim et al., Vet. Immunol. Immunopath. 74: 315-327, 2000, Chew et al., Anim. Feed Sci. Tech. 59:103-114, 1996, Cerveny et al., FASEB J. 13 :A210. 1999; Brown et al., FASEB J. 15: A954, 2001), and is also with similar results higher mitogen-induced splenocyte proliferative response in mice and dogs.

Natural killer cells serve in an immuno-surveillance capacity against tumors. Therefore, the observed enhancement of NK cell cytotoxic activity with dietary astaxanthin suggests that this ketocarotenoid may play a role in cancer etiology. Others have reported increased cytotoxic T lymphocyte activity and IFN-γ production in astaxanthin-fed mice (Jyonouchi et al., Nutr. Cancer 36: 59-65, 2000). Similarly, we reported that dietary lutein increased IFN-γ mRNA expression but decreased the expression of IL-10 in splenocytes of tumor-bearing mice; these changes paralleled the inhibitory action of lutein against tumor growth (Cerveny et al., FASEB J. 13:A210, 1999).

The increased B cell population and PWM-induced lymphocyte proliferative response with dietary astaxanthin indicate heightened humoral immunity. In mice, astaxanthin also increased the ex vivo antibody response of splenocytes to T-cell antigens (Jyonouchi et al., Nutr. Cancer 21: 47-58, 1994).

Astaxanthin may function to protect circulating blood cells through its antioxidant action (Martin et al., J. Prakt. Chem. 341-: 302-308, 1999; Naguib, J. Agric. Food Chem. 48: 1150-1154, 2000). In fact, astaxanthin was approximately 100 fold more protective than lutein and β-carotene against UVA-induced oxidative stress in vitro (O'Connor and O'Brien, J. Dermatol. Sci. 16: 226-230, 1998). Why dietary astaxanthin did not reduce lipid peroxidation as measured by changes in iso-prostane concentrations is unclear, especially when others have reported that astaxanthin was more effective than β-carotene and vitamin E in inhibiting lipid peroxidation.

A startling observation from this study is the dramatic decrease in DNA damage in subjects who received astaxanthin. This protection was observed by 4 weeks of feeding. In addition, maximal response was observed with 2 mg astaxanthin. This represents the first report on the protective effect of astaxanthin against DNA damage using the plasma 8-OHdG as the marker.

### EXAMPLE 2

### Obtaining a Natural Astaxanthin-Enriched Extract

This example provides one method for obtaining a natural astaxanthin-enriched extract from H. pluvialis, using supercritical CO₂ extraction, which extract is useful in the methods described herein.

By way of example, commercially available dried and ground Haematococcus algae meal is procured. Producers of such meal can be found, for instance, in Hawaii, Israel, India, and Sweden (for instance, Cyanotech Corp., Kailua-Kona, Hawaii; Algatechnolgies (1998) Ltd., Elat, Israel; Fuji Chemical Industries, Toyama, Japan; AstaReal AB, Gustavsberg, Sweden; Microalgal Biotechnology, Sede-Boker, Israel). The algal meal is extracted in a supercritical fluid extraction facility using CO₂. By way of examples, stainless steel baskets are filled with algal meal and placed into a high pressure extraction vessel. Clean food-grade carbon dioxide (without chemical co-solvents or entrainers) in the supercritical state is passed through the extraction baskets, to load astaxanthin, other carotenoids, and lipids from the algal meal into the CO₂. The “loaded” carbon dioxide passes through a back-pressure regulator into a separation vessel under lower pressure and temperature, to transfer the carbon dioxide into the gas phase and separate it from the astaxanthin-enriched carotenoid oleoresin. The extract is then drawn off from the separation vessel through a valve and collected in, for instance, portable stainless steel vessels. Beneficially, the carbon dioxide can be recovered and recycled. Fully extracted (spent) algal meal (which may have been extracted more than once with CO₂) is removed from the basket to complete the extraction process.

Astaxanthin-complex carotenoid oleoresin collected form the separation vessel can be analyzed for astaxanthin and other carotenoid content, and then packaged in sealed airtight food-grade containers (for instance, made from HDPE). It is optimally stored at low temperature (e.g., 2-10° C.)

This disclosure demonstrates that oral administration of astaxanthin, particularly in the form of a natural astaxanthin-enriched extract, is highly effective at reducing oxidative DNA damage in healthy humans. The disclosure further provides methods of applying astaxanthin, or a preparation comprising astaxanthin, to subjects in order to reduce, inhibit, prevent, or otherwise decrease oxidative DNA damage in a cell of the subject. It will be apparent that the precise details of the methods described may be varied or modified without departing from the spirit of the described invention. We claim all such modifications and variations that fall within the scope and spirit of the claims below.

1. A method of reducing, preventing, ameliorating, or reversing oxidative DNA damage in a subject, comprising orally administering a therapeutically effective dose of a natural astaxanthin extract to the subject, whereby the natural astaxanthin extract reduces, prevents, ameliorates, or reverses the oxidative DNA damage.

2. The method of claim 1, wherein the natural astaxanthin extract comprises predominantly mono- and di-ester forms of astaxanthin.

3. The method of claim 2, wherein the natural astaxanthin extract comprises no more than about 5% free astaxanthin, about 45-50% astaxanthin monoesters, about 10-40% astaxanthin diesters, and other carotenoids in the remaining percentage.

4. The method of claim 3, wherein the other carotenoids comprise β-carotene, lutein, canthaxanthin, or a mixture of two or more thereof.

5. The method of claim 1, wherein the natural astaxanthin extract is derived from yeast or microalgae.

6. The method of claim 5, wherein the natural astaxanthin extract is derived from Haematococcus pluvialis.

7. The method of claim 1, wherein the astaxanthin in the extract is greater than 95% (3S,3′S) astaxanthin.

8. The method of claim 7, wherein the astaxanthin in the extract is about 100% (3S,3′S) astaxanthin.

9. The method of claim 1, wherein the astaxanthin in the extract comprises about 55-62% E-astaxanthin, about 13-18% 9Z-astaxanthin, and about 23-29% 13Z-astxanthin.

10. The method of claim 1, wherein the natural astaxanthin extract further comprises fatty acids, and the fatty acids are one or more of Lauric, Tridecanoic, Myristic, Pentadecanoic, Palmitic, cis-9-Palmitoleic, Heptadecanoic, cis-10-Heptadecenoic, Stearic, cis-9-Oleic and/or trans-9-Elaidic, cis-9,12-Linoleic and/or trans-9,12-Linolelaidic, Arachidic, alpha-Linolenic, cis-11-Eicosenoic, Linolenic, Heneicosanoic, cis-11,14-Eicosadienoic, Behenic, cis-8,11,14-Eicosatrienoic, cis-13-Erucic, cis-11,14,17-Eicosatrienoic, cis-5,8,11,14-Arachidonic, and cis-5,8,11,14,17-Eicosapentaenoic acids.

11. The method of claim 5, wherein the natural astaxanthin extract is derived from a Phaffia species.

12. The method of claim 1, wherein the natural astaxanthin extract is produced by a process comprising supercritical carbon dioxide extraction.

13. The method of claim 1, wherein the natural astaxanthin extract is administered to the subject in combination with at least one additional biologically active compound.

14. The method of claim 13, wherein the biologically active compound is a carotenoid, an antioxidant, a vitamin, or a second natural extract.

15. The method of claim 1, wherein the natural astaxanthin extract is:
dissolved in oil;
dispersed in oil;
dispersed in an aqueous medium;
homogenized in an aqueous medium;
encapsulated;
processed into dry material; or
a combination of two or more thereof.

16. The method of claim 15, wherein the natural extract is processed into dry material, and the form of the dry material is stabilized beadlets, a powder, a granule, or a combination of two or more thereof.

17. The method of claim 1, wherein the natural astaxanthin extract is formulated as a liquid, a liquid capsule, a solid capsule or a tablet.

18. The method of claim 1, wherein the natural antioxidant extract is administered to the subject in a food or beverage product.

19. The method of claim 1, wherein the therapeutically effective dose of astaxanthin reduces the oxidative DNA damage by at least 30% compared to a subject not administered the therapeutically effective dose of astaxanthin.

20. The method of claim 1, wherein the subject is human.

21. The method of claim 1, wherein the oxidative DNA damage comprises oxidative DNA damage in immune cells.

22. The method of claim 21, wherein the immune cells are cells, B-cells, monocytes, neutrophils, natural killer cells, splenocytes, or a mixture of two or more thereof.

23. The method of claim 1, wherein the therapeutically effective dose is about 0.5-1000 mg astaxanthin per day.

24. The method of claim 23, wherein the therapeutically effective dose is about 1-10 mg per day.

25. The method of claim 1, wherein the therapeutically effective dose is about 2 mg per day; about 4 mg per day, or about 8 mg per day.

26. (canceled)

27. (canceled)
